(19) United States
(12) Patent Application Publication
(10) Pub. No.: US 2015/0284504 A1
(43) Pub. Date: Oct. 8, 2015

(54) HETEROCYCLIC COMPOUNDS AND THE SYNTHESIS METHOD THEREOF

(71) Applicant: National Chiao Tung University, Hsinchu (TW)

(72) Inventors: Yen-Ju Cheng, Hsinchu (TW); Sheng-Wen Cheng, Hsinchu (TW); De-Yang Chiou, Hsinchu (TW)

(21) Appl. No.: 14/506,379

(22) Filed: Oct. 3, 2014

(30) Foreign Application Priority Data
Apr. 2, 2014 (TW) 103112382

(51) Int. Cl. C08G 61/12; H01L 51/00; C07D 495/04

(52) U.S. Cl. CPC C08G 61/126; C07D 495/04; H01L 51/0043; H01L 51/0036; H01L 51/42

(57) ABSTRACT

A synthesis method of a heterocyclic compound is disclosed. The synthesis method includes steps of: carrying out a McMurry coupling reaction on a first compound having a carbonyl group to form a second compound, wherein the second compound includes an alkyl group which is symmetrical to a symmetrical center, and carrying out a 6π-cyclization on the second compound to form a third compound.

### CROSS REFERENCE TO RELATED APPLICATIONS

The application claims the benefit of the Taiwan Patent Application No. 103112382, filed on Apr. 2, 2014, in the Taiwan Intellectual Property Office, the disclosures of which are incorporated herein in their entirety by reference.

### FIELD OF THE INVENTION

The present invention relates to a compound and a synthesis method thereof and, more particularly, to a heterocyclic compound and a synthesis method thereof.

### BACKGROUND OF THE INVENTION

The acenedithiophene (AcDT) family has been used as key building blocks to make superior organic semiconductors. First, α-positions of the two terminal thiophene rings of AcDTs can be selectivity functionalized for easy, versatile π-extension and precise polymerization. The rigid and coplanar structures of AcDTs can facilitate π-electron delocalization and π-π stacking for achieving efficient charge transport. Second, the aromatic size and molecular shape of AcDTs can chemically manipulate the electronic and steric properties. Conjugated polymers comprising tricyclic benzodithiophene (BDT) or pentacyclic anthradithiophene (ADT) derivatives can be used to produce high efficient organic field effect transistors (OFETs) and polymer solar cells (PSCs).

Because of these advantages, recently another promising and attractive AcDT, tetracyclic naphthodithiophene (NDT), has received popular attention to development new organic semiconductors. Coplanar and rigid NDT has a naphthalene ring at the center and two fused thiophene rings at the ends. Especially, the molecular packing of NDT-based polymers is strongly influenced by the geometry of NDTs. NDT family contains different regioisomers including linear-fused NDT (lNDT) and angular-fused NDT (aNDT). Moreover, depending on the geometry of the fused thiophenes, the sulfur atoms in aNDT can be functionalized on either the •- or •-positions of the central naphthalene moiety, yielding two regioisomers denoted as -aNDT and -aNDT, respectively.

However, the rigid and coplanar aNDT have no aliphatic side chains as solubilizing groups, which severely hinders the wet process ability of non-alkylted aNDT-based oligomers and polymers and limits their PSCs and OFETs device performance.

Since 2009 to 2011, Takimiya et al. had reported the synthesis of non-alkylated or 2,7-alkylated α-aNDT/α-aNDS, which used 2,6-dihydroxynaphthalene as the starting material and then underwent chlorination, Sonogashira coupling reaction and cyclization using Na₂S or NaBH₄/Se. They also proposed the synthesis of non-alkylated or 2,7-alkylated •-aNDT, which used the initiator 2,6-dibromo-1,5-dihydroxynaphthalene and then underwent Sonogashira coupling reaction and cyclization using Na₂S.

However, both of two synthetic routes given above are not suitable for cross coupling polymerization. Because the polymers based on non-alkylated α-aNDT, α-aNDS or •-aNDT will possess poor solubility. Moreover, 2,7-alkylated α-aNDT/α-aNDS aren't able to polymerize at their 2,7-positions due to the alkylated sites.

Recently, a useful approach to selectively functionalize two alkyl chains at the 5, 10-positions of •-aNDT was also developed by Takimiya et al. in 2012. However, introducing the side chains into 5,10-positions of •-aNDT to improve the solubility of the resulting polymers could imposes a negative effect on the effective conjugation and molecular stacking due to the steric hindrance-induced twisting between the neighboring units. This might result in the lower current (J_{sc}) of PSCs.

In 2013, Li et al. published the polymers based on 4,9-dialkoxyl •-aNDT. They successfully used 1,5-dihydroxynaphthalene as the starting material to synthesize α-aNDT with two alkoxyl side chains at 4- and 9-positions. However, the electron-donating alkoxy groups could raise HOMO energy level of the corresponding polymers, which could lower the open circuit voltage (V_{oc}) and the performance in PSCs.

As mentioned above, it is known that using iridium catalyst can introduce the side chains into 5- and 10-positions of α-aNDT, which is suitable for polymerization. Recently, a D-A copolymer was polymerized by reacting 5,10-didodecyl •-aNDT monomer with dibromodithiophenyl-NT monomers (DTNT). The corresponding PSCs performance exhibited a high PCE of 8.2%.

Using 1,5-dihydroxynaphthalene as a starting material to synthesize 4,9-dialkoxyl α-aNDT, which was polymerized with two electron acceptors, DTBT and DTBO, to obtain polymer PzNDTDTBT and PzNDTDTBO. The performance in PSCs are moderate power efficiency of 3.2% and 5.1%, respectively, as result of the lower V_{oc}.

The side-chain steric and electronic effects at 4,9- and 5,10-positions could play a crucial role in determining the photophysical, orbital, and bulk properties which are worthy of systematic investigation. Compared to dialkylation at outer 5,10-positions, substitution at inner 4,9-positions could in principle reduce their steric interference with other alkyl groups on the neighboring aromatic rings, thereby maintaining coplanar backbone of the resulting oligomers or polymers. Therefore, introducing the alkyl side chains into 4- and 9-positions of •-aNDT is a more ideal and promising way to enhance the solubility, the intramolecular packing and charge transportation of the polymers.

In order to overcome the drawbacks in the prior art, heterocyclic compounds and a synthesis method thereof are disclosed. The particular design in the present invention not only solves the problems described above, but also is easy to implement. Thus, the present invention has great utility for the industry.

### SUMMARY OF THE INVENTION

The present invention discloses a new synthesis method, which uses McMurry coupling reaction, Sonogashira coupling reaction and 6π-cyclization. The synthesis method offers an easy way to form the regiospecific products which are heterocyclic compounds. This synthesis method also can control the position of the alkyl side chains and hetero atoms (such as sulfur, oxygen, nitrogen, and selenium) in the heterocyclic compounds. Thus, four geometric isomers for each type of heterocyclic compounds can be constructed and soluble in common organic solvents. The synthesis method further discloses an appropriate ratio to mix the heterocyclic compounds and specific electron acceptors (such as DTBT, DTFBT, DPP and FIT, which are further described below) to form p-type semiconductors. The blends with the p-type materials and the n-type materials form the active layer of PSCs.

In accordance with one aspect of the present invention, a heterocyclic compound is disclosed. The heterocyclic compound is represented by one selected from a group consisting of formulas (1)-(4):

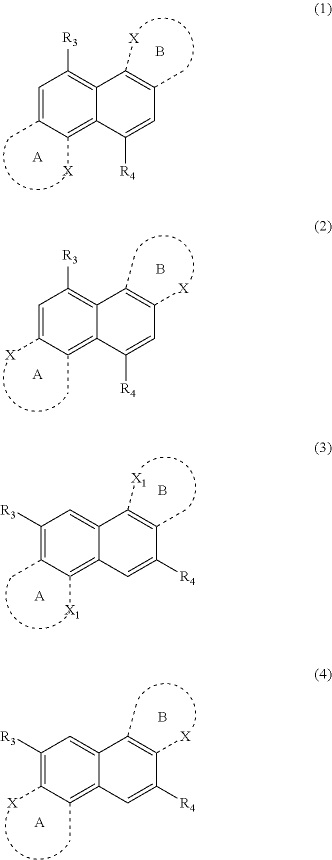

wherein: either of R₃ and R₄ is one of C₁₋₃₀ linear saturated alkyl group and C₁₋₃₀ branched saturated alkyl group; either of A and B is one of C₃₋₈ unsaturated aromatic ring and C₃₋₈ unsaturated heteroaromatic ring, wherein the C₃₋₈ unsaturated heteroaromatic ring includes at least one heteroatom; X is the respective heteroatom being one selected from a group consisting of nitrogen (N), sulfur (S), oxygen (O) and selenium (Se); X₁ is the respective heteroatom being one selected from a group consisting of N, S, O and Se, where if R₃ and R₄ are both one of C₁₂ and C₁₆ linear saturated alkyl groups, X₁ is the heteroatom being one selected from a group consisting of N, O and Se; R₃ and R₄ are symmetrical to a symmetrical center; and formulas (1)-(4) are isomers of one another.

In accordance with another aspect of the present invention, a synthetic method of a heterocyclic compound is disclosed. The synthetic method includes steps of: carrying out a McMurry coupling reaction on a first compound having a carbonyl group to form a second compound, wherein the second compound includes an alkyl group which is symmetrical to a symmetrical center; and carrying out a 6π-cyclization on the second compound to form a third compound.

In accordance with a further aspect of the present invention, a heterocyclic compound prepared by the method according to the present invention is disclosed. The heterocyclic compound is represented by one selected from a group consisting of the following formulas (13)-(16):

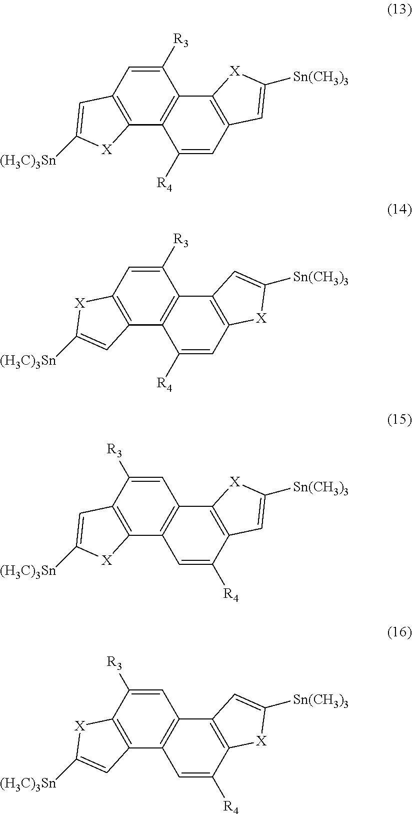

wherein: either of R3 and R4 is represented by one of the following formulas (5)-(10):

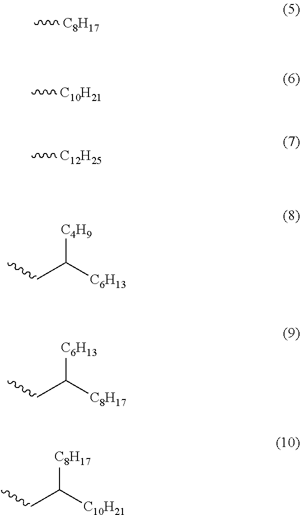

X is one selected from a group consisting of N, S, O, and Se; and the compounds of formulas (13)-(16) are isomers of one another.

In accordance with a further aspect of the present invention, a heterocyclic compound prepared by the method according to the present invention is disclosed. The heterocyclic compound is represented by one of the following formulas (17) and (18):

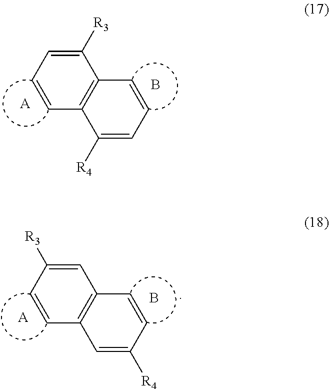

In accordance with a further aspect of the present invention, a synthesis method of a heterocyclic compound is disclosed. The synthesis method includes steps of: carrying out a 6π-cyclization on a first compound having a heterocyclic ring, an alkenyl group and an alkynyl group to form a second compound, wherein the second compound includes a fused heterocyclic ring having at least one alkyl substituent.

In addition to being used in the solar industry, the heterocyclic compound of the present invention can also be used in the fields of organic optics sensors and flexible displays industry. Furthermore, the synthesis method of heterocyclic compounds disclosed by the present invention can synthesize four isomers with fused heteroaromatic rings by controlling the positions of hetero atoms and the alkyl side chains into the heterocyclic compounds.

The objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows the heterocyclic compound structures according to a first embodiment of the present invention;

FIG. 2 shows the synthetic routes of the heterocyclic compound according to a second embodiment of the present invention;

FIGS. 3(a) and 3(b) show the synthetic routes of the heterocyclic compound according to a third embodiment of the present invention;

FIGS. 4(a) and 4(b) show the synthetic routes of the heterocyclic compound according to the third embodiment of the present invention; and

FIG. 5 shows the UV-Vis absorbance spectra of heterocyclic compounds with different configurations according to a fourth embodiment of the present invention.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

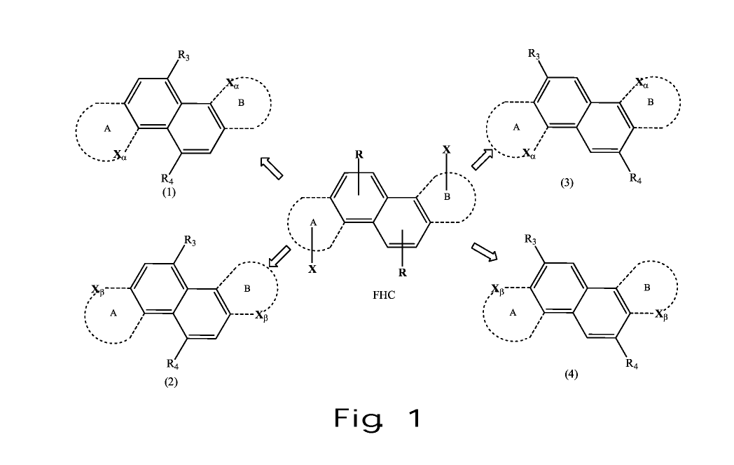

Please refer to FIG. 1, which shows the heterocyclic compound structure according to a first embodiment of the present invention. Heterocyclic compound FHC is the general formula of the present invention, which is a fused heterocycle having an alkyl side chain R. As shown in FIG. 1, the structures are formed by a naphthalene ring, a first additional ring A and a second additional ring B which are symmetrical to the center of symmetry, wherein the first additional ring A and the second additional ring B are on two sides of the naphthalene ring. The naphthalene ring includes two alkyl groups R, R3 and R4. The alkyl groups R, R3 and R4 are symmetrical to the center of symmetry. Both the additional rings A and B include a hetero atom X, X_{α} and X_{β}. By configuring the hetero atom X, X_{α} and X_{β} at different positions on the additional rings, the heterocyclic compounds (1), (2), (3) and (4) having the same molecular formula but different configurations can be formed, wherein the heterocyclic compound (1) is in the first configuration, the heterocyclic compound (2) is in the second configuration, the heterocyclic compound (3) is in the third configuration, and the heterocyclic compounds (4) is in the fourth configuration.

Both the first additional ring A and the second additional ring B are selected from one of a C₃₋₈ unsaturated aromatic ring and a C₃₋₈ unsaturated heteroaromatic ring, wherein the C₃₋₈ unsaturated heteroaromatic ring includes at least one hetero atom X, X_{α} and X_{β}, and the hetero atom X, X_{α} and X_{β} includes at least one of nitrogen (N), sulfur (S), oxygen (O) and selenium (Se). R, R3 and R4 are selected from C₁₋₃₀ linear saturated alkyl group or C₁₋₃₀ branched saturated alkyl group.

According to one embodiment of the present invention, the first additional ring A and the second additional ring B have the same structure, and R3 and R4 have the same structure.

According to one embodiment of the present invention, the first additional ring A and the second additional ring B are selected from the following structural formulas:

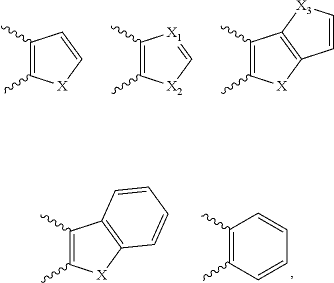

wherein X includes at least one of N, S, O and Se; X₁ includes N; X₂ includes at least one of N, S and O; and X₃ includes S.

According to one embodiment of the present invention, R3 and R4 are selected from the following structural formulas:

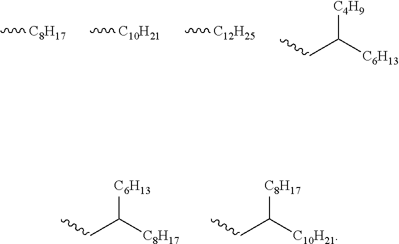

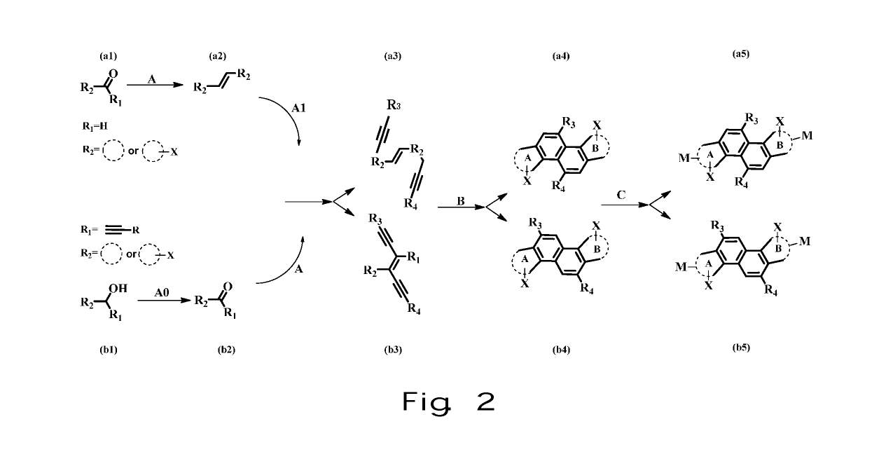

Please refer to FIG. 2, which shows the synthesis routes of the heterocyclic compound according to a second embodiment of the present invention. The synthesis method includes the following steps: carrying out a McMurry coupling reaction A (titanium tetrachloride/zinc (TiCl₄/Zn) with solvent: pyridine (pyridine), tetrahydrofuran (THF)) to compounds (a1), (b2) having a carbonyl group, to form compounds (a2), (b3), wherein the compound (a2) includes the alkyl group R2 and the compound (b3) includes alkyl groups R3, R4 which are symmetrical to the center of symmetry; carrying out a 6π-cyclization B (1,8-diazabicyclo[5.4.0]undec-7-ene (DBU), with solvent: N-methylpyrrolidone (NMP), by reflux) to compounds (a3), (b3), to form compounds (a4), (b4).

In the structure of compounds (b2) (COR₁R₂) having a carbonyl group, R₁ is an unsaturated alkyl group including C₃₋₃₂ linear chain or branched chain, wherein when R₁ is selected from one of C₃₋₃₂ linear chain and branched chain, R₁ includes a C≡C bond connected to the carbonyl group in the general formula. R2 is selected from C₃₋₈ unsaturated aromatic rings or C₃₋₈ unsaturated heteroaromatic ring, wherein the C₃₋₈ unsaturated heteroaromatic ring includes at least one hetero atom. The hetero atom includes at least one of N, S, O and Se. The C₃₋₈ unsaturated heteroaromatic ring further includes a substituent. The substituent is selected from one of hydrogen (H) and halogen group, wherein the halogen group is selected from one of bromine (Br) and iodine (I).

Furthermore, in the compound (a1), when R is H, R2 is selected from one of C₃₋₈ unsaturated aromatic rings and C₃₋₈ unsaturated heteroaromatic ring, and R2 further includes a halogen as a substituent group, carrying out McMurry coupling reaction A to the compound (a1) will form an intermediate (a2). After carrying out a Sonogashira coupling reaction A1 (—C≡CR3, —C≡CR4, cuprous iodide (CuI), bis(triphenylphosphine) palladium dichloride/triphenylphosphine (PdCl₂ (PPh₃)₂/PPh₃, catalyst), with solvent: DIPA/THF) to the intermediate (a2), the compound (a3) will be synthesized.

The compound (b2) can be prepared by performing a pretreatment, i.e. carrying out a pyridinium chlorochromate (PCC) oxidation reaction A0 to synthesize the compound (b2), wherein R1 is C≡CR (R is R3, R4), and R2 is selected from one of a C₃₋₈ unsaturated aromatic ring and a C₃₋₈ unsaturated heteroaromatic ring.

The structure of compounds (a4), (b4) is formed by a naphthalene ring, a first additional ring A and a second additional ring B which are symmetrical to the center of symmetry, wherein the first additional ring A and the second additional ring B are on two sides of the naphthalene ring. The naphthalene ring includes two alkyl groups R3 and R4, and the alkyl groups R3 and R4 are symmetrical to the center of symmetry. Both the additional rings A and B include a hetero atom X, wherein compounds (a4), (b4) can form two isomers by configuring the hetero atom X at different positions of the additional rings A and B.

According to one embodiment of the present invention, the structures of compounds (a4), (b4) are both formed by a naphthalene ring, a first additional ring A and a second additional ring B which are symmetrical to the center of symmetry, wherein the first additional ring A and the second additional ring B are on two sides of the naphthalene ring. The naphthalene ring includes two alkyl groups R3 and R4, and the alkyl groups R3 and R4 are symmetrical to the center of symmetry. Both additional rings A and B are benzene rings.

According to one embodiment of the present invention, R3 and R4 are selected from the following structural formulas:

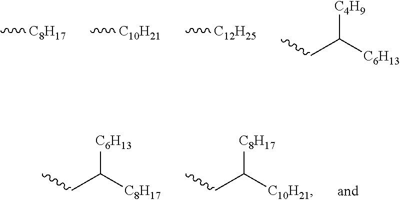

the additional rings A and B are selected from the following structural formulas:

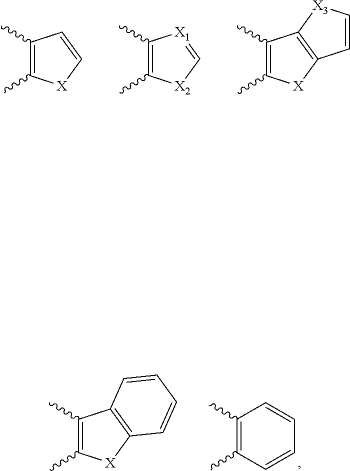

wherein X includes at least one of N, S, O and Se; X₁ includes N; X₂ includes one of N, S and O; and X₃ includes S.

As shown in FIG. 2, carrying out a stannylation reaction on compounds (a4), (b4) will synthesize compounds (a5), (b5). Both the compounds (a5), (b5) include at least one substituent M. The substituent M is Sn(CH₃)₃ or Sn(butyl)₃. The stannylation reaction is carried out with one of trimethyltin chloride (Me₃SnCl) and tributyltin chloride ([butyl]₃SnCl) in the presence of one of n-butyllithium (n-BuLi) and lithium diisopropylamide (LDA).

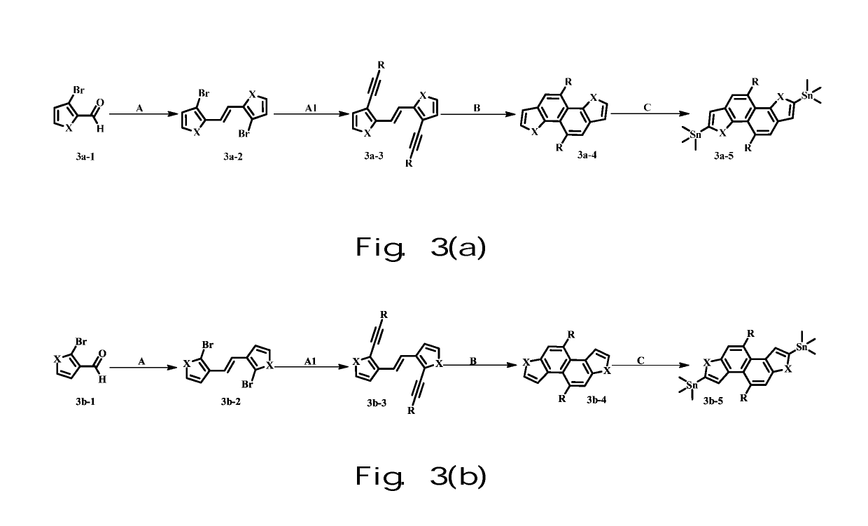

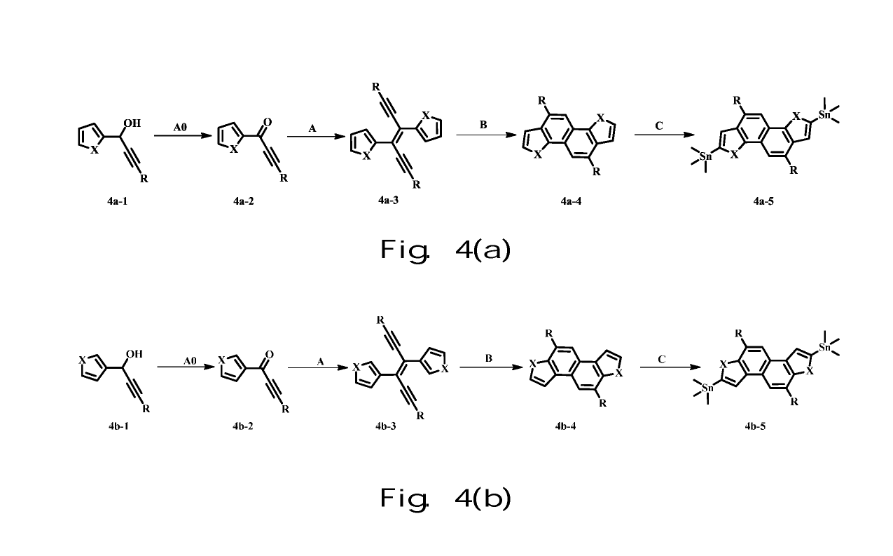

Please refer to FIG. 3 (a), FIG. 3(b), FIG. 4(a) and FIG. 4(b), which show the synthetic routes of the heterocyclic compound according to a third embodiment of the present invention. In this embodiment, the heterocyclic compounds in different configurations having the additional ring A and additional ring B, which are both C₄ unsaturated heteroaromatic rings, are synthesized. The reaction processes include the following four types.

Please refer to FIG. 3 (a), which uses compound 3a-1 as starting material to carry out McMurry coupling reaction A (TiCl₄/Zn, with solvent: pyridine (pyridine), tetrahydrofuran (THF)) to form compound 3a-2, and then carry out Sonogashira coupling reaction A1 on the compound 3a-2 to form a carbon-carbon triple bond (i.e. —C≡CR3, —C≡CR4) so as to form compound 3a-3. Next, 6π-cyclization B on the compound 3a-3 is carried out to cause the 3a-3 to have a heterocyclic ring, an alkenyl group and an alkynyl group to form compound 3a-4. Then, a stannylation reaction on compound 3a-4, n-butyl lithium (n-BuLi) and trimethyltin chloride (Me₃SnCl) is carried out to obtain compound 3a-5, wherein the compound 3a-4 has the first configuration.

Please refer to FIG. 3(b), which uses compound 3b-1 as starting material to carry out McMurry coupling reaction A (TiCl₄/Zn, with solvent: pyridine (pyridine), tetrahydrofuran (THF)) to form compound 3b-2, and then Sonogashira coupling reaction A1 on the compound 3b-2 is carried out to form carbon-carbon triple bond (i.e. —C≡CR3, —C≡CR4) so as to form compound 3b-3. Next, 6π-cyclization B on the compound 3b-3 is carried out to cause the compound 3b-3 with a heterocyclic ring, an alkenyl group and an alkynyl group to form compound 3b-4. Then, a stannylation reaction on compound 3b-4, n-butyl lithium (n-BuLi) and trimethyltin chloride (Me₃SnCl) is carried out to obtain compound 3b-5, wherein the compound 3b-4 has the second configuration.

Referring to FIG. 4(a), which uses compound 4a-1 as starting material to carry out oxidation reaction A0 (Pyridinium chlorochromate (PCC), with solvent: dichloromethane (CH₂Cl₂)) to cause the hydroxyl group to be carbonyl group to form compound 4a-2, and then McMurry coupling reaction A is carried out to form compound 4a-3. Next, 6π-cyclization B on the compound 4a-3 is carried out to cause the compound 4b-3 with a heterocyclic ring, an alkenyl group and an alkynyl group to form compound 4b-4. Then, a stannylation reaction on compound 4a-4, n-butyl lithium (n-BuLi) and trimethyltin chloride (Me₃SnCl) is carried out to obtain compound 4a-5, wherein the compound 4a-4 has the third configuration.

Referring to FIG. 4(b), which uses compound 4b-1 as starting material to carry out oxidation reaction A0 (Pyridinium chlorochromate (PCC), with solvent: dichloromethane (CH₂Cl₂)) to cause the hydroxyl group to carbonyl group to form compound 4b-2, and then McMurry coupling reaction A is carried out to form compound 4b-3. Next, 6π-cyclization B on the compound 4b-3 is carried out to cause the compound 4b-3 with heterocyclic ring, an alkenyl group and an alkynyl group to form compound 4b-4. Then, stannylation on compound 4b-4, n-butyl lithium (n-BuLi) and trimethyltin chloride (Me₃SnCl) is carried out to obtain compound 4b-5, wherein the compound 4b-4 has the fourth configuration.

In the reaction processes above, in the compound, R is selected from one of C₁₋₃₀ linear chain and C₁₋₃₀ branched chain saturated alkyl groups, and X is selected from one of N, S, O and Se.

In the reaction processes above, the synthesized compound 3a-4, compound 3b-4, compound 4a-4 and compound 4b-4 are isomers.

According to one embodiment of the present invention, in the compound, R is selected from one of C₁₂ and C₂₀ branched chain saturated alkyl groups, and X is S.

According to the reaction processes above, when carrying out structure characterization on compound 3a-4, compound 3b-4, compound 4a-4 and compound 4b-4 (R is C₁₀ linear chain saturated alkyl groups, and X is S), ¹H and ¹³C nuclear magnetic resonance spectroscopy (Nuclear Magnetic Resonance (NMR), in which D-chloroform (deuterated chloroform) is used as a solvent, the chemical shift unit is ppm, and ¹HNMR uses δ=0.00 ppm (TMS) or 7.26 ppm (D-CHCl₃) as internal reference, while ¹³CNMR uses δ=77.00 ppm (D-CHCl₃) as an internal reference) and mass spectrometry, which uses EI or FAB as ionization methods (not shown), have confirmed that the present invention synthesizes four isomers of heterocyclic compounds. Figures of X-ray crystal structures of compound 3a-4 and compound 3b-4 (not shown) show that the configuration of the side chain and the geometric shape of the conjugated architecture have significant influence on intermolecular stacking, wherein in compound 3a-4, C₁₀ linear chain saturated alkyl groups are configured on two sides of two x-x stacking channels, and in compound 3b-4, C₁₀ linear chain saturated alkyl groups are configured between two x-x stacking channels.

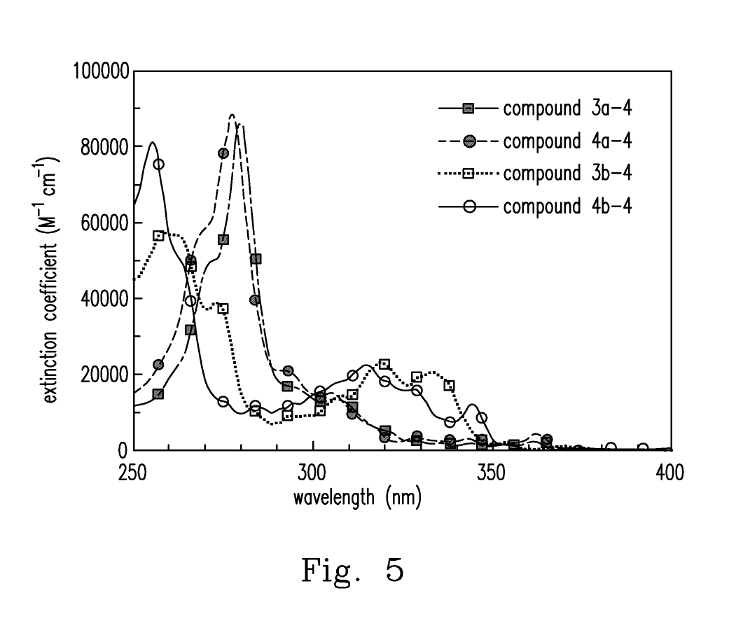

The results of absorption spectral experiments on the above compound is shown in FIG. 5. Referring to FIG. 5, the absorption range of the compound was 250-350 nm. Compared with compound 3a-4 and compound 4a-4, compound 3b-4 and compound 4b-4 have a blue shift phenomenon (moving towards short wavelengths) in the wavelength range between 260-270 nm; in addition, they have significant absorption peaks in the wavelength range between 300-350 nm. The alkyl groups in the compound influence the performance of the optical characteristics. That is, compared to compounds 4a-4 and 4b-4, compounds 3a-4 and 3b-4 have the of slight red shift phenomenon. Therefore, this result shows the influence of optical characteristics from different configurations, and so the present invention, which can synthesize four heterocyclic compounds of four different configurations successfully, is proven.

By carrying out electrochemical analysis on compound 3a-4, compound 3b-4, compound 4a-4 and compound 4b-4 (R is C₁₀ linear saturated alkyl groups, and X is S), the HOMO energy levels of compound 3a-4, compound 3b-4, compound 4a-4 and compound 4b-4 of −5.66, −5.60, −5.70 and −5.63 eV were calculated, respectively. Due to a lower HOMO energy level, a better antioxidant capability and a higher open circuit voltage in a photovoltaic element can be obtained. Compared with compound 3b-4 and compound 4b-4, compound 3a-4 and compound 4a-4 have a lower HOMO energy level. Therefore, with the configurations of compound 3a-4 and compound 4a-4, the element efficiency and stability can be effectively improved.

In the reaction processes above, the synthesized compound 3a-5, compound 3b-5, compound 4a-5 and compound 4b-5 are all electron donors, which can carry out a copolymerization with a specific electron acceptor via a microwave reactor by a first specific value to form a low-bandgap conjugated macromolecule.

The specific electron acceptor is selected from one of 2(bromothiophenyl)N-(2-ethylhexyl)-pyrrolopyrrole-dione (Br-DPP), 3-fluoro-2-[(2-ethylhexyl)carbonyl]dibromothiophenylthiophene (Br-FIT), bis(bromothiophenyl)-2,1,3-benzothiadiazole (Br-DTBT), bis(hexylbromothiophenyl)-2,1,3-benzothiadiazole (Br-C8-DTBT) and bis(hexylbromothiophenyl)difluoro-2,1,3-benzothiadiazole (Br-C8-DTFBT).

The compound (such as 3a-5, 3b-5, 4a-5 or 4b-5) and the electron donor have a first specific ratio. The first specific ratio is a molar ratio, wherein the ratio of the compound to the electron donor is one to a first specific value. The first specific value is one.

As shown in FIGS. 3(a), 3(b), 4(a) and 4(b), unlike R in compound 4a-4 and compound 4b-4, which is configured in the 5- and 10-positions (outer) of the naphthalene ring of the compounds, R in compound 3a-4 and compound 3b-4 is configured in the 4- and 9-positions (inner) of the naphthalene ring of the compounds. This contributes to the coplanarity and the intermolecular stacking of the low band gap conjugated polymer formed when the compounds (electron donor) are bound to a specific electron acceptor. Because R is close to the inner side, its smaller steric hindrance between the adjacent electron acceptor can cause the structure of the donor and the acceptor to be more coplanar. This is beneficial to the intermolecular stacking. Therefore, if an active layer material includes the low band gap conjugated polymer, the element made thereby will have a better electronic delivery channel, an improved current and improved efficiency.

The low-bandgap conjugated polymer is mixed with a fullerene derivative to form active layer for an organic solar cell, wherein the fullerene derivative is selected from one of PC₆₁BM and PC₇₁BM. The low-bandgap conjugated polymer and the fullerene derivative have a second specific ratio. The second specific ratio is a weight percentage, wherein the ratio of the polymer to the fullerene derivative is one to a second specific value. The range of the specific value is 0.5-2.0.

According to one embodiment of the present invention, the low-bandgap conjugated polymer is selected from the following structural formulas:

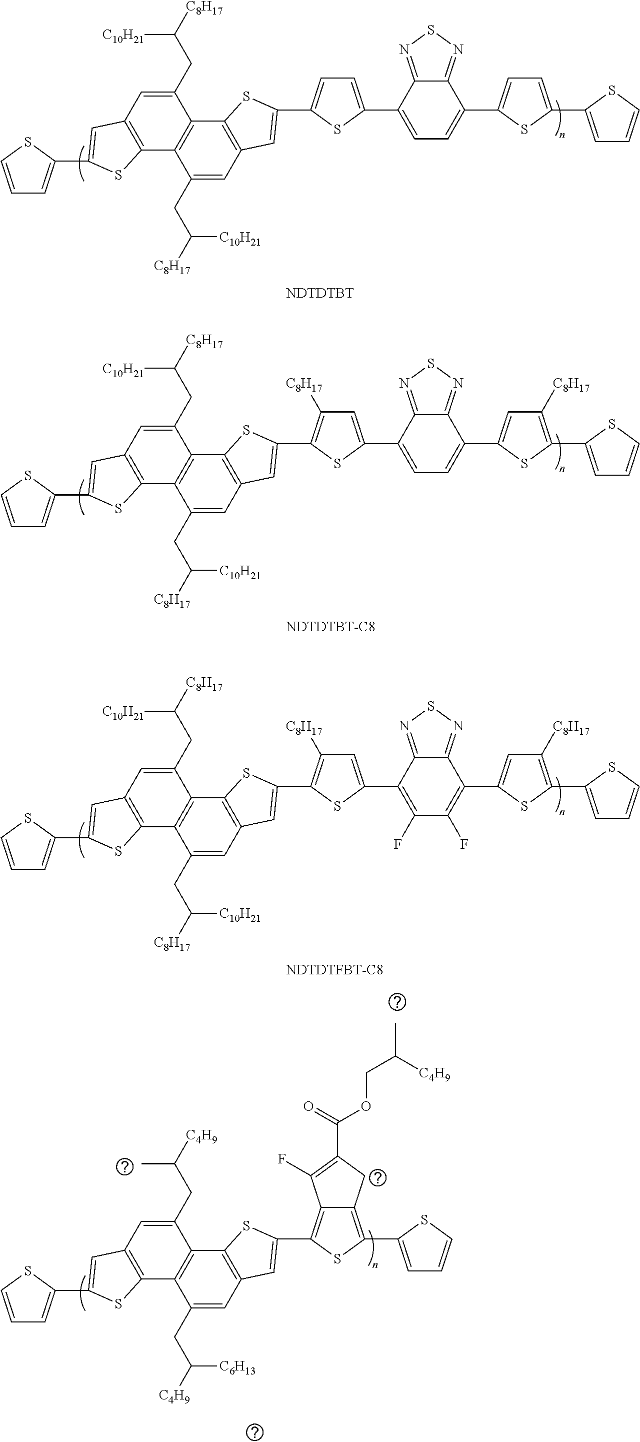

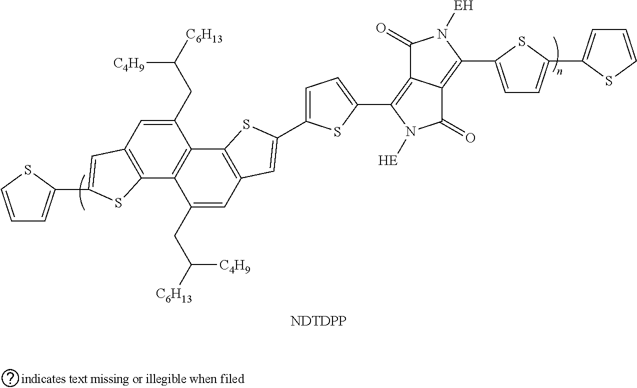

wherein n is the number of the repeating unit, the range of n is between 1-50, and the electron donors in the low-bandgap conjugated polymer are all in the first configuration.

Thermal property analysis on NDTDTBT-C8 and NDTDTFBT-C8 (not shown) was carried out, wherein the thermal property analysis used a thermogravimetric analyzer (TGA) (not shown) and a differential scanning calorimeter (DSC). Based on the experimental results from the TGA, it can be seen that the measured low-bandgap conjugated polymers all have the thermal decomposition temperature (T_{d}) of 450° C., meaning that they all have good thermal stability and can be used in the manufacturing process for PSCs. In addition, based on the experimental results from the DSC, it can be seen that the measured low-bandgap conjugated polymers all have the melting point (Tₘ) and the crystallization point (T_{c}), showing that the polymers have good intramolecular stacking and semi-crystalline natures.

For the optical property analysis of NDTDTBT-C8 and NDTDTFBT-C8 (not shown), based on the results of the UV-Vis absorption spectrum, it can be seen that regardless of whether the low-bandgap conjugated polymers are in the solution or in the solid state, they have the broad absorption range between 300˜700 nm.

When using the low-bandgap conjugated polymers and the fullerene derivative as the materials of the active layer to manufacture an organic solar cell device, the following steps are included: forming a conventional configuration device (anode/hole transport layer/active layer/electron transport layer/cathode (ITO/PEDOT:PSS (4083)/active layer/Ca/Al)), wherein in the active layer, the blend ratio of the low-bandgap conjugated polymers to the fullerene derivative is 1.5:1, 1:1, 1:1.5 or 1:2. The choice of solvents includes chlorobenzene (CB) and o-dichlorobenzene (o-DCB). The active layer is coated on the hole transport layer by spin coating, wherein the spin speed range is between 800-1400 rpm. In the process of preparing elements, the solvent-annealing step (solvent-annealing, SA) or the thermal annealing step (thermal-annealing, TA) is used to regulate the morphology in the active layer.

According to one embodiment of the present invention, the experimental condition is: conventional configuration device, CB, 1000 rpm and without SA or TA. Through the solar simulator (AM 1.5 G 100 mW/cm²) (not shown), it can be seen that when using NDTDTFBT-C8 as a p-type material of the active layer, the power conversion efficiency (PCE) is 6.52% (the open circuit voltage (V_{oc}) is 0.84 V, the short circuit current (J_{sc}) is −11.28 mA/cm₂, the fill factor (ff) is 68.8%, and the HOMO energy level is −5.59 eV). Based on this experiment, it can be seen that compared to the HOMO energy level (PCE of PzNDTDTBT is 3.22%, V_{oc} is 0.6 V and the HOMO energy level is −5.15; PCE of PzNDTDTBO is 5.07%, V_{oc} is 0.74 V and the HOMO energy level is −5.30) of the NDT-based macromolecule having the alkyl carbon chain (PzNDTDTBT and PzNDTDTBO, wherein the electron donor is in the first configuration), the NDT-based polymers having alkyl chain of the present invention have a lower HOMO energy level. This indicates that the sites of the alkyl chain help reduce the HOMO energy level and raise the V_{oc} value, demonstrating more excellent power conversion efficiency.

In conclusion, the present invention discloses a novel synthesis method which can easily synthesize four isomeric heterocyclic compounds having alkyl side chain. The synthesis method of the present invention can control the position of the alkyl chain and hetero atoms in heterocyclic compounds, wherein the heterocyclic compounds with introduced alkyl chains can improve the solubility of the polymer (making the material easy to process devices), and improve the morphology and stability of the active layer at the same time. The heterocyclic compound in the first configuration is thus synthesized successfully. The synthesis method solved the problem that the alkyl chain without an oxygen atom cannot be introduced to a specific position (the heterocyclic compound in the first configuration) Also, the HOMO energy levels of the polymers are effectively reduced to achieve high V_{oc}, while the coplanar of the polymer chain is maintained to increase intermolecule stacking. This achieves good carrier mobility and J_{sc}, thereby enhancing the efficiency of polymer solar cells.

### EMBODIMENTS

1. A heterocyclic compound represented by one selected from a group consisting of formulas (1)-(4):

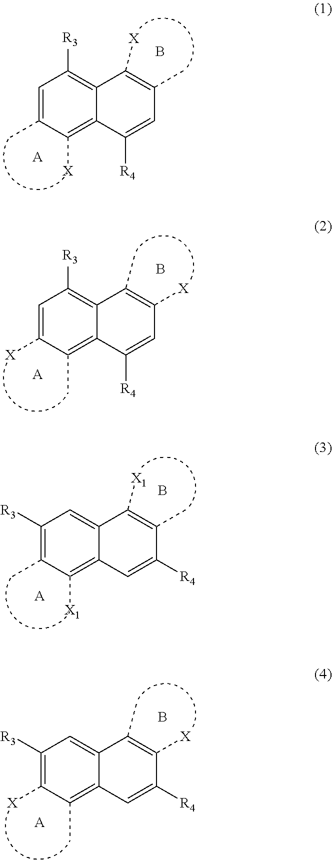

wherein: either of R₃ and R₄ is one of C₁₋₃₀ linear alkyl group and C₁₋₃₀ branched saturated alkyl group; either of A and B is one of C₃₋₈ unsaturated aromatic ring and C₃₋₈ unsaturated heteroaromatic ring, wherein the C₃₋₈ unsaturated heteroaromatic ring includes at least one heteroatom; X is the respective heteroatom being one selected from a group consisting of nitrogen (N), sulfur (S), oxygen (O) and selenium (Se); X₁ is the respective heteroatom being one selected from a group consisting of N, S, O and Se, where if R₃ and R₄ are both one of C₁₂ and C₁₆ linear saturated alkyl groups, X₁ is the respective heteroatom being one selected from a group consisting of N, O and Se; R₃ and R₄ are symmetrical to a symmetrical center, and formulas (1)-(4) are isomers of one another.
2. The heterocyclic compound of Embodiment 1, wherein either of R3 and R4 is one selected from a group consisting of the following formulas (5)-(10):

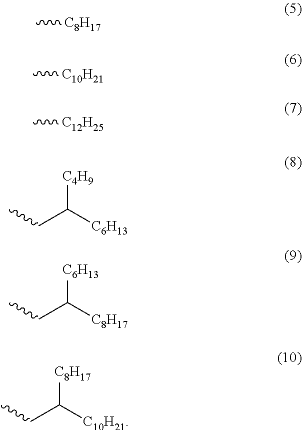

3. A synthesis method of a heterocyclic compound, comprising steps of: carrying out a McMurry coupling reaction on a first compound having a carbonyl group to form a second compound, wherein the second compound includes an alkyl group which is symmetrical to a symmetrical center, and carrying out a 6π-cyclization on the second compound to form a third compound.
4. The synthesis method of Embodiment 3, wherein the first compound is represented by the following formula (11):

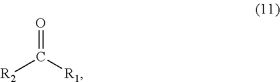

wherein: R₁ is one selected from a group consisting of hydrogen (H), C₃₋₃₂ linear unsaturated alkyl groups and C₃₋₃₂ branched unsaturated alkyl groups, where when R₁ is one of C₃₋₃₂ linear unsaturated alkyl groups and C₃₋₃₂ branched unsaturated alkyl groups, R₁ includes a C≡C bond connected to the carbonyl group in the formula (11); R2 is one of C₃₋₈ unsaturated aromatic ring and C₃₋₈ unsaturated heteroaromatic ring, wherein the C₃₋₈ unsaturated heteroaromatic ring includes at least one heteroatom selected from a group consisting one of N, S, O and Se; and either of the C₃₋₈ unsaturated aromatic ring and the C₃₋₈ unsaturated heteroaromatic ring further includes a substituent being one of hydrogen (H) and halogen group, wherein the halogen group is one of bromine (Br) and iodine (I).
5. The synthesis method of any one of Embodiments 3-4, further comprising steps of: carrying out the McMurry coupling reaction on the first compound to form an intermediate; and carrying out a Sonogashira coupling reaction on the intermediate to form the second compound.
6. The synthesis method of any one of Embodiments 3-5, further comprising steps of: performing a pretreatment including a pyridinium chlorochromate (PCC) oxidation reaction on a secondary alcohol compound to form the first compound.
7. The synthesis method of any one of Embodiments 3-6, wherein the third compound has four isomers and is represented by the following formula (12):

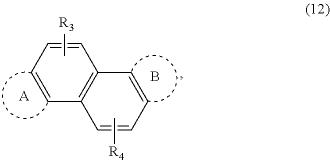

wherein: either of R3 and R4 is one of C₁₋₃₀ linear saturated alkyl group and C₁₋₃₀ branched saturated alkyl group; either of A and B is one of C₃₋₈ unsaturated aromatic ring and Cu unsaturated heteroaromatic ring, wherein the C₃₋₈ unsaturated heteroaromatic ring includes at least one heteroatom selected from a group consisting of N, S, O and Se; and R₃ and R₄ are symmetrical to a symmetrical center.
8. The synthesis method of any one of Embodiments 3-7, further comprising steps of: causing the third compound to carry out a stannylation reaction with one of trimethyltin chloride (Me₃SnCl) and tributyltin chloride ([butyl]₃SnCl) in the presence of one of n-butyllithium (n-BuLi) and lithium diisopropylamide (LDA) to form a fourth compound, and the fourth compound comprises at least one substituent being one of Sn(CH₃)₃ and Sn(butyl)₃.
9. The heterocyclic compound prepared by the synthesis method of any one of embodiments 3-8, the heterocyclic compound is represented by one selected from a group consisting of the following formulas (13)-(16):

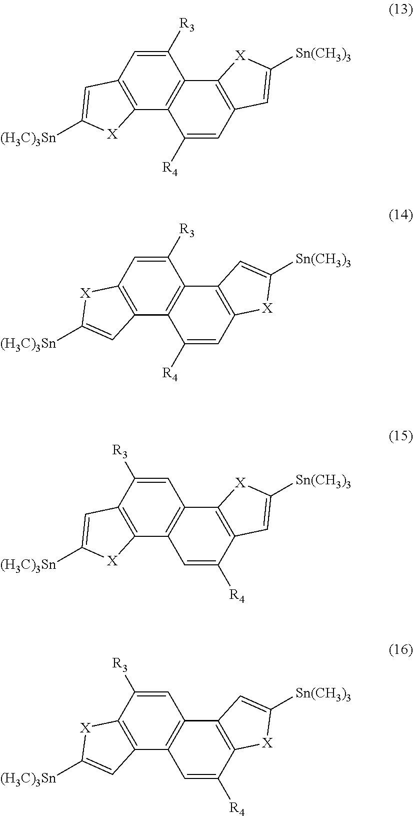

wherein: either of R3 and R4 is represented by one of the following formulas (5)-(10):

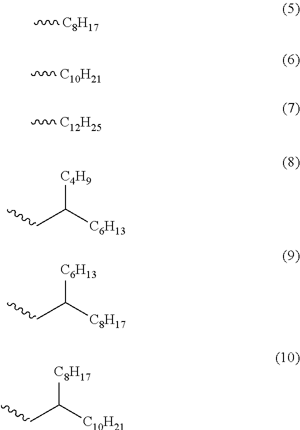

X is one selected from a group consisting of N, S, O, and Se; and the compounds of formulas (13)-(16) are isomers of one another.
10. The heterocyclic compound prepared by the synthesis method of any one of Embodiments 3-8, the heterocyclic compound is represented by one of the following formulas (17) and (18):

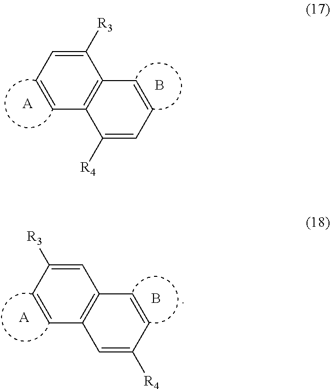

11. The heterocyclic compound of any one of Embodiments 9-10, wherein: either of R₃ is R₄ are represented by one selected from a group consisting of the following formulas (5)-(10):

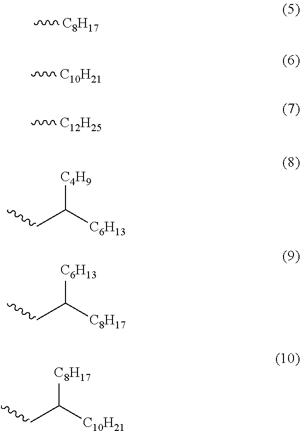

either of A and B is represented by one selected from a group consisting of the following formulas (19)-(23):

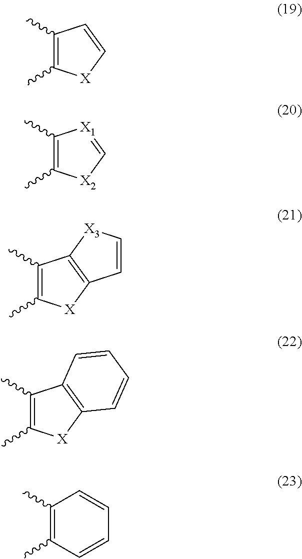

X comprises at least one selected from a group consisting of N, S, O and Se; X₁ comprises N; X₂ comprises at least one selected from a group consisting of N, S and O; X₃ comprises S; and R₃ and R₄ symmetrize at a symmetric center; and A and B further comprises a substituent being one of Sn(CH₃)₃ and Sn(n-butyl)₃.
12. The heterocyclic compound of any one of Embodiments 9-11, wherein: when using the heterocyclic compound in an all solution wet-process, R₃ and R₄ determine a solubility of the heterocyclic compound in the all solution wet-process.
13. The heterocyclic compound of any one of Embodiments 9-12, wherein: the heterocyclic compound is an electron donor carrying out a copolymerization with a specific electron acceptor via a microwave reactor to form a low-bandgap conjugated polymer, the specific electron acceptor is selected from a group consisting of 2(bromothiophenyl)N-(2-ethylhexyl)-pyrrolopyrrole-dione (Br-DPP), 3-fluoro-2-[(2-ethylhexyl)carbonyl]dibromothiophenylthiophene (Br-FIT), bis(bromothiophenyl)-2,1,3-benzothiadiazole (Br-DTBT), bis(hexylbromothiophenyl)-2,1,3-benzothiadiazole (Br-C8-DTBT) and bis(hexylbromothiophenyl)difluoro-2,1,3-benzothiadiazole (Br-C8-DTFBT); the heterocyclic compound and the specific electron acceptor have a first specific ratio of 1 to a first specific value, wherein the first specific ratio is a molar ratio, and the first specific value is 1; the low-bandgap conjugated polymer is mixed with a fullerene derivative to form an active layer of an organic thin-film solar cell, wherein the fullerene derivative is one of PC₆₁BM and PC₇₁BM; and the low-bandgap conjugated polymer and the fullerene derivative have a second specific ratio of 1 to a second specific value, wherein the second specific ratio is a weight percentage, and the range of the second specific value is 0.5-2.0.
14. A synthesis method of a heterocyclic compound, comprising steps of: carrying out a 6π-cyclization on a first compound having a heterocyclic ring, an alkenyl group and an alkynyl group to form a second compound, wherein the second compound includes a fused heterocyclic ring having at least one alkyl substituent.
15. The synthesis method of Embodiment 14, further comprising steps of: carrying out a McMurry coupling reaction on a compound having a carbonyl group to form the first compound.
16. The synthesis method of any one of Embodiments 14-15, wherein the compound having the carbonyl group is represented by the following formula (11):

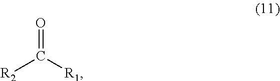

wherein: R₁ is one selected from a group consisting of hydrogen (H), C₃₋₃₂ linear unsaturated alkyl groups and C₃₋₃₂ branched unsaturated alkyl groups, provided that when R1 is one of C₃₋₃₂ linear unsaturated alkyl groups and C₃₋₃₂ branched unsaturated alkyl groups, R₁ includes a C≡C bond connected to the carbonyl group in the formula (11); R₂ is one of C₃₋₈ unsaturated aromatic ring and C₃₋₈ unsaturated heteroaromatic ring, wherein the C₃₋₈ unsaturated heteroaromatic ring includes at least one heteroatom being one selected from a group consisting of N, S, O and Se; and either of the C₃₋₈ unsaturated aromatic ring and the C₃₋₈ unsaturated heteroaromatic ring further includes a substituent being one of hydrogen (H) and halogen group, wherein the halogen group is one of bromine (Br) and iodine (I).
17. The synthesis method of any one of Embodiments 14-16, further comprising steps of: carrying out the McMurry coupling reaction on the compound having the carbonyl group to form an intermediate; and carrying out a Sonogashira coupling reaction on the intermediate to form the first compound.
18. The synthesis method of any one of Embodiments 14-17, further comprising steps of: performing a pretreatment including a pyridinium chlorochromate (PCC) oxidation reaction on a secondary alcohol compound to form the compound having the carbonyl group.
19. The synthesis method of any one of Embodiments 14-18, wherein the second compound has four isomers and is represented by the following formula (12):

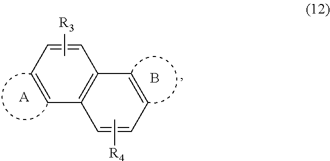

wherein: either of R₃ and R₄ is one of C₁₋₃₀ linear saturated alkyl group and C₁₋₃₀ branched saturated alkyl group; either of A and B is one of C₃₋₈ unsaturated aromatic ring and C₃₋₈ unsaturated heteroaromatic ring, wherein the C₃₋₈ unsaturated heteroaromatic ring includes at least one heteroatom selected from a group consisting of N, S, O and Se; and R3 and R4 are symmetrical to a symmetrical center.
20. The synthesis method of any one of Embodiments 14-19, further comprising steps of: causing the second compound to carry out a stannylation reaction with one of trimethyltin chloride (Me₃SnCl) and tributyltin chloride ([butyl]₃SnCl) in the presence of one of n-butyllithium (n-BuLi) and lithium diisopropylamide (LDA) to form a third compound, and the third compound comprises at least one substituent being one of Sn(CH₃)₃ and Sn(butyl)₃.

1. A heterocyclic compound represented by one selected from a group consisting of formulas (1)-(4): 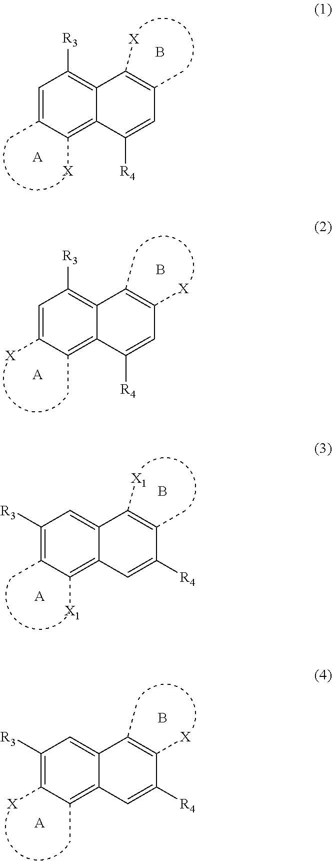
wherein:
either of R₃ and R₄ is one of C₁₋₃₀ linear saturated alkyl group and C₁₋₃₀ branched saturated alkyl group;
either of A and B is one of C₃₋₈ unsaturated aromatic ring and C₃₋₈ unsaturated heteroaromatic ring, wherein the C₃₋₈ unsaturated heteroaromatic ring includes at least one heteroatom;
X is the respective heteroatom being one selected from a group consisting of nitrogen (N), sulfur (S), oxygen (O) and selenium (Se);
X, is the respective heteroatom being one selected from a group consisting of N, S, O and Se, where if R₃ and R₄ are both one of C₁₂ and C₁₆ linear saturated alkyl groups, X₁ is the respective heteroatom being one selected from a group consisting of N, O and Se;
R₃ and R₄ are symmetrical to a symmetrical center, and
formulas (1)-(4) are isomers of one another.

2. The heterocyclic compound according to claim 1, wherein either of R₃ and R₄ is one selected from a group consisting of the following formulas (5)-(10):

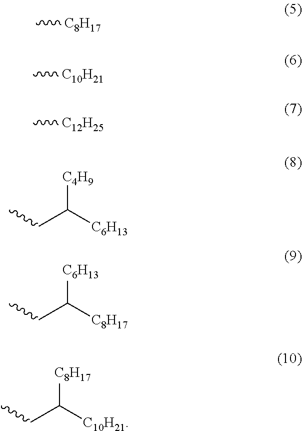

3. A synthesis method of a heterocyclic compound, comprising steps of:
carrying out a McMurry coupling reaction on a first compound having a carbonyl group to form a second compound, wherein the second compound includes an alkyl group which is symmetrical to a symmetrical center, and
carrying out a 6π-cyclization on the second compound to form a third compound.

4. The synthesis method according to claim 3, wherein the first compound is represented by the following formula (11): 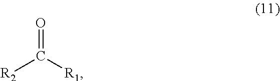
wherein:
R₁ is one selected from a group consisting of hydrogen (H), C₃₋₃₂ linear unsaturated alkyl groups and C₃₋₃₂ branched unsaturated alkyl groups, where when R₁ is one of C₃₋₃₂ linear unsaturated alkyl groups and C₃₋₃₂ branched unsaturated alkyl groups, R₁ includes a C≡C bond connected to the carbonyl group in the formula (11);
R₂ is one of C₃₋₈ unsaturated aromatic ring and C₃₋₈ unsaturated heteroaromatic ring, wherein the C₃₋₈ unsaturated heteroaromatic ring includes at least one heteroatom selected from a group consisting one of N, S, O and Se; and
either of the C₃₋₈ unsaturated aromatic ring and the C₃₋₈ unsaturated heteroaromatic ring further includes a substituent being one of hydrogen (H) and halogen group, wherein the halogen group is one of bromine (Br) and iodine (I).

5. The synthesis method according to claim 4, further comprising steps of:
carrying out the McMurry coupling reaction on the first compound to form an intermediate; and
carrying out a Sonogashira coupling reaction on the intermediate to form the second compound.

6. The synthesis method according to claim 4, further comprising steps of: performing a pretreatment including a pyridinium chlorochromate (PCC) oxidation reaction on a secondary alcohol compound to form the first compound.

7. The synthesis method according to claim 3, wherein the third compound has four isomers and is represented by the following formula (12): 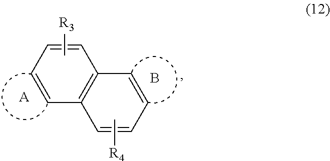
wherein:
either of R₃ and R₄ is one of C₁₋₃₀ linear saturated alkyl group and C₁₋₃₀ branched saturated alkyl group;
either of A and B is one of C₃₋₈ unsaturated aromatic ring and C₃₋₈ unsaturated heteroaromatic ring, wherein the C₃₋₈ unsaturated heteroaromatic ring includes at least one heteroatom selected from a group consisting of N, S, O and Se; and
R₃ and R₄ are symmetrical to a symmetrical center.

8. The synthesis method according to claim 7, further comprising steps of:
causing the third compound to carry out a stannylation reaction with one of trimethyltin chloride (Me₃SnCl) and tributyltin chloride ([butyl]₃SnCl) in the presence of one of n-butyllithium (n-BuLi) and lithium diisopropylamide (LDA) to form a fourth compound, and the fourth compound comprises at least one substituent being one of Sn(CH₃)₃ and Sn(butyl)₃.

9. The heterocyclic compound prepared by the synthesis method according to claim 8, the heterocyclic compound is represented by one selected from a group consisting of the following formulas (13)-(16): 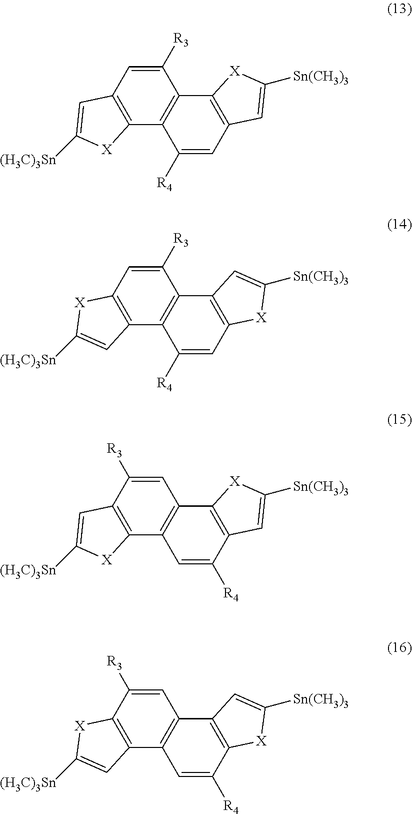
wherein:
either of R₃ and R₄ is represented by one of the following formulas (5)-(10): 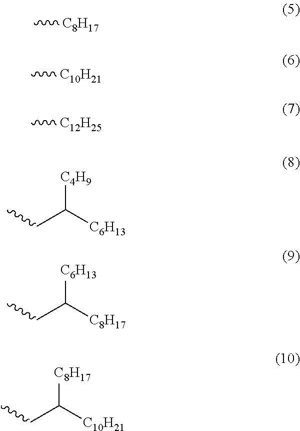
X is one selected from a group consisting of N, S, O, and Se; and
the compounds of formulas (13)-(16) are isomers of one another.

10. The heterocyclic compound prepared by the synthesis method according to claim 8, the heterocyclic compound is represented by one of the following formulas (17) and (18):

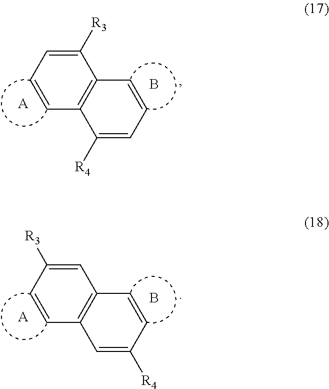

11. The heterocyclic compound according to claim 10, wherein:
either of R₃ is R₄ are represented by one selected from a group consisting of the following formulas (5)-(10): 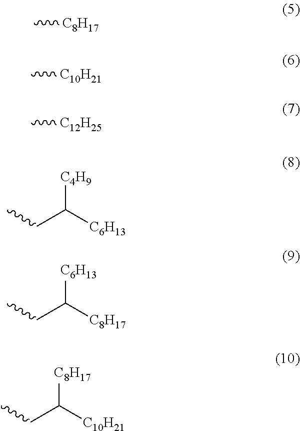
either of A and B is represented by one selected from a group consisting of the following formulas (19)-(23): 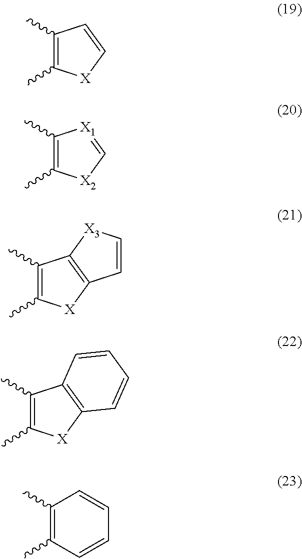
X comprises at least one selected from a group consisting of N, S, O and Se;
X₁ comprises N;
X₂ comprises at least one selected from a group consisting of N, S and O;
X₃ comprises S; and
R₃ and R₄ symmetrize at a symmetric center; and
A and B further comprises a substituent being one of Sn(CH₃)₃ and Sn(n-butyl)₃.

12. The heterocyclic compound according to claim 11, wherein:
when using the heterocyclic compound in an all solution wet-process, R₃ and R₄ determine a solubility of the heterocyclic compound in the all solution wet-process.

13. The heterocyclic compound according to claim 11, wherein:
the heterocyclic compound is an electron donor carrying out a copolymerization with a specific electron acceptor via a microwave reactor to form a low-bandgap conjugated polymer, the specific electron acceptor is selected from a group consisting of 2(bromothiophenyl)N-(2-ethylhexyl)-pyrrolopyrrole-dione (Br-DPP), 3-fluoro-2-[(2-ethylhexyl)carbonyl]dibromothiophenylthiophene (Br-FTT), bis(bromothiophenyl)-2,1,3-benzothiadiazole (Br-DTBT), bis(hexylbromothiophenyl)-2,1,3-benzothiadiazole (Br-C8-DTBT) and bis(hexylbromothiophenyl)difluoro-2,1,3-benzothiadiazole (Br-C8-DTFBT); the heterocyclic compound and the specific electron acceptor have a first specific ratio of 1 to a first specific value, wherein the first specific ratio is a molar ratio, and the first specific value is 1; the low-bandgap conjugated polymer is mixed with a fullerene derivative to form an active layer of an organic thin-film solar cell, wherein the fullerene derivative is one of PC₆₁BM and PC₇₁BM; and the low-bandgap conjugated polymer and the fullerene derivative have a second specific ratio of 1 to a second specific value, wherein the second specific ratio is a weight percentage, and the range of the second specific value is 0.5-2.0.

14. A synthesis method of a heterocyclic compound, comprising steps of:
carrying out a 6π-cyclization on a first compound having a heterocyclic ring, an alkenyl group and an alkynyl group to form a second compound, wherein the second compound includes a fused heterocyclic ring having at least one alkyl substituent.

15. The synthesis method according to claim 14, further comprising steps of:
carrying out a McMurry coupling reaction on a compound having a carbonyl group to form the first compound.

16. The synthesis method according to claim 15, wherein the compound having the carbonyl group is represented by the following formula (11): 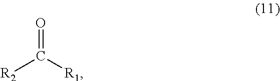
wherein:
R₁ is one selected from a group consisting of hydrogen (H), C₃₋₃₂ linear unsaturated alkyl groups and C₃₋₃₂ branched unsaturated alkyl groups, provided that when R₁ is one of C₃₋₃₂ linear unsaturated alkyl groups and C₃₋₃₂ branched unsaturated alkyl groups, R₁ includes a C≡C bond connected to the carbonyl group in the formula (11);
R₂ is one of C₃₋₈ unsaturated aromatic ring and C₃₋₈ unsaturated heteroaromatic ring, wherein the C₃₋₈ unsaturated heteroaromatic ring includes at least one heteroatom being one selected from a group consisting of N, S, O and Se; and
either of the C₃₋₈ unsaturated aromatic ring and the C₃₋₈ unsaturated heteroaromatic ring further includes a substituent being one of hydrogen (H) and halogen group, wherein the halogen group is one of bromine (Br) and iodine (I).

17. The synthesis method according to claim 16, further comprising steps of:
carrying out the McMurry coupling reaction on the compound having the carbonyl group to form an intermediate; and
carrying out a Sonogashira coupling reaction on the intermediate to form the first compound.

18. The synthesis method according to claim 16, further comprising steps of:
performing a pretreatment including a pyridinium chlorochromate (PCC) oxidation reaction on a secondary alcohol compound to form the compound having the carbonyl group.

19. The synthesis method according to claim 14, wherein the second compound has four isomers and is represented by the following formula (12): 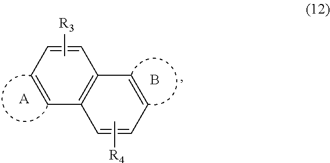
wherein:
either of R₃ and R₄ is one of C₁₋₃₀ linear saturated alkyl group and C₁₋₃₀ branched saturated alkyl group;
either of A and B is one of C₃₋₈ unsaturated aromatic ring and C₃₋₈ unsaturated heteroaromatic ring, wherein the C₃₋₈ unsaturated heteroaromatic ring includes at least one heteroatom selected from a group consisting of N, S, O and Se; and
R₃ and R₄ are symmetrical to a symmetrical center.

20. The synthesis method according to claim 19, further comprising steps of:
causing the second compound to carry out a stannylation reaction with one of trimethyltin chloride (Me₃SnCl) and tributyltin chloride ([butyl]₃SnCl) in the presence of one of n-butyllithium (n-BuLi) and lithium diisopropylamide (LDA) to form a third compound, and the third compound comprises at least one substituent being one of Sn(CH₃)₃ and Sn(butyl)₃.
